(19) United States
(12) Patent Application Publication
(10) Pub. No.: US 2005/0187556 A1
(43) Pub. Date: Aug. 25, 2005

(54) Universal percutaneous spinal access system

(75) Inventors: Richard S. Stack, Chapel Hill, NC (US); Michael S. Williams, Santa Rosa, CA (US)

Correspondence Address: DEANNA J. SHIRLEY, 3418 BALDWIN WAY, SANTA ROSA, CA, 95403, US

(73) Assignee: SyneCor, LLC

(21) Appl. No.: 11/057,001

(22) Filed: Feb. 9, 2005

Related U.S. Application Data
(60) Provisional application No. 60/547,929, filed on Feb. 25, 2004.

(51) Int. Cl. A61B 17/32; A61F 2/46; A61F 2/44

(52) U.S. Cl. 606/79

(57) ABSTRACT

A system for accessing the interior of a vertebral body or the intervertebral disc space above or below the vertebral body is disclosed. The system comprises a steerable cutting means for creating a path within or through the vertebral body to allow access for other devices to deliver a therapy, and may be housed within a flexible catheter shaft. The steerable cutting means may create a path to allow access to a device for removing tissue, and/or additional a devices for delivering a therapy, such as, for example, a filling material and/or a prosthesis. A method of accessing the interior of a vertebral body or the intervertebral disc space using the system, and a method of treatment of spinal disorders using the system are also disclosed.

### RELATED APPLICATIONS

This application is related to and claims the benefit of the priority date of U.S. Provisional Patent Application Ser. No. 60/547,929 to Stack, et al., entitled “Universal Percutaneous Spinal Access System”, filed Feb. 25, 2004.

### FIELD OF THE INVENTION

The invention herein relates generally to medical devices and methods of treatment, and more particularly to devices and methods used to deliver treatment to the spine.

### BACKGROUND OF THE INVENTION

Intervertebral disc degeneration, and disc and vertebral trauma are leading causes of pain and disability, occurring in a substantial majority of people at some point during adulthood. The intervertebral disc, comprising primarily the nucleus pulposus and surrounding annulus fibrosus, constitutes a vital component of the functional spinal unit. The intervertebral disc maintains space between adjacent vertebral bodies, absorbs impact between and cushions the vertebral bodies. Deterioration of the biological and mechanical integrity of an intervertebral disc as a result of disease and/or aging may limit mobility and produce pain, either directly or indirectly as a result of disruption of the functioning of the spine. Trauma induced damage may include ruptures, tears, prolapse, herniations, and other injuries that cause pain and reduce strength and function.

As an example of common vertebral trauma, approximately 700,000 vertebral fractures occur annually among an estimated 44 million Americans suffering from osteoporosis. Other disease processes, including tumor growth, especially round cell tumors, avascular necrosis, and defects arising from endocrine conditions also result in a weakened condition and/or fractures. Such other conditions, whether in the vertebrae or at other sites, are also causes of significant pain and reduced mobility in patients. Estimated health care costs of treating back pain in the United States exceed $60 billion annually, and pose substantial costs in the form of disability payments, workers' compensation and lost wages.

Non-operative therapeutic options for individuals with neck and back pain include rest, analgesics, physical therapy, heat, and manipulation. These treatments fail in a significant number of patients. Current surgical options for spinal disease include discectomy, discectomy combined with fusion, and fusion alone. Numerous discectomies are performed annually in the United States. The procedure is effective in promptly relieving significant radicular pain, but, in general, the return of pain increases proportionally with the length of time following surgery. In fact, the majority of patients experience significant back pain by ten years following lumbar discectomy. Similarly, the insertion of pins and other devices into diseased vertebrae may prove unsatisfactory. All surgical options are invasive, and lead to significant hospitalization and recovery time.

In addition to intervertebral disc disease and injury, osteoporosis is a significant health issue around the world. Osteoporosis, literally “porous” bone, is a disease characterized by low bone mass and density, and structural deterioration of bone tissue. Osteoporosis leads to bone fragility; increased susceptibility to fractures including compression fractures; neural compression; insufficient vertical support by the spine; and pain. According to the National Osteoporosis Foundation, osteoporosis is a major public health threat for an estimated 44 million Americans. According to the International Osteoporosis Foundation, osteoporosis is responsible for more than 1.5 million fractures annually, including approximately 700,000 vertebral fractures, as well as numerous fractures of the hip, wrist, and other sites.

Vertebral fractures are the most common osteoporotic fracture. Approximately 20-25% of women over the age of 50 have one or more vertebral fractures. Once a woman suffers a first vertebral fracture, the shift in force transmission upon all vertebrae result in a five-fold increase in the risk of developing a new fracture within one year. Vertebral fractures, like hip fractures, are associated with a substantial increase in mortality among otherwise relatively healthy older women. Following such fractures, treatment that requires attachment of pins, screws, or similar devices to the vertebral bodies may not be feasible because of the underlying instability of the diseased bone. Osteoporosis and vertebral fractures are further characterized by decreased height, and often collapse, of the vertebral bodies. Such decrease leads to stooped posture, decreased lung capacity, impaired mobility, neural compression, and pain.

Consequently, there is a need in the art to treat vertebral and intervertebral injury and disease using minimally invasive techniques. Further, there is a need for a system that allows access to either or both the vertebral body or the disc space above and/or below the vertebral body.

### SUMMARY OF THE INVENTION

A system for use in the treatment of spinal disorders is disclosed. The system may comprise a steerable guide catheter, a means for penetrating a vertebral body, and a flexible catheter for extraction of tissue and/or delivery of therapy. The therapy may comprise a filling material, a prosthesis, or both. The treatment may be partial or complete discectomy, partial or total disc replacement, vertebroplasty, vertebral decompression, prosthesis delivery, and prosthesis deployment. The means for penetrating the vertebral body may comprise a vibrating tip, ultrasound, a high speed burr or a flexible drill. The system may also comprise a pressure regulator to control the delivery of a therapy, which may comprise, for example, a curable polymeric foam or, as an additional example, other curable polymer.

A method for obtaining access to the interior of a vertebral body or to the intervertebral body disc space is also disclosed. The method may comprise accessing the interior of a vertebral body from a transpedicular approach, and may be followed by the step of creating a path within the vertebral body. The path may be generally oriented along the horizontal plane within the vertebral body, or generally oriented in the vertical plane. The method may include the additional step of entering the intervertebral disc space either above or below the vertebral body.

A method for treating a spinal disorder comprising obtaining access to the interior of a vertebral body from a transpedicular approach is also disclosed. The method may further comprise creating a path within said vertebral body. The path may be generally oriented in the horizontal plane within the vertebral body, or generally oriented in the vertical plane within the vertebral body. The method may further comprise the steps of delivering a prosthesis within the vertebral body, and deploying the prosthesis within the vertebral body. It may also include the step of introducing a filling material within the vertebral body.

The method may further comprise the steps of entering the intervertebral disc space, extracting some or all of the native disc, and/or delivering and deploying a prosthesis to the intervertebral disc space. The step of deploying the prosthesis may comprise introducing a filling material into the prosthesis.

### BRIEF DESCRIPTION OF THE DRAWINGS

FIG. 1 is a “see-through” side view of a vertebral body illustrating the introduction of a device that is a part of a system according to the invention into the vertebral body.

FIG. 2 is a “see-through” plan view of a vertebral body illustrating an alternative path created within the vertebral body according to the invention.

### DETAILED DESCRIPTION OF THE INVENTION

“Vertebroplasty” is a procedure used to augment diseased and/or fractured vertebral bodies, in which a biocompatible cement or filling material is infused into the vertebral body through a large bore needle under fluoroscopic guidance.

“Kyphoplasty” is a procedure similar to vertebroplasty, with the added step of creating space within the vertebral body and restoring vertebral height with the use of a balloon prior to injecting biocompatible cement or filling material.

“Spinal unit” refers to a set of the vital functional parts of the spine including a vertebral body, endplates, facets, intervertebral space and intervertebral disc.

The phrase “decompressing the bone” refers to a process during treatment according to the invention by which a collapsed portion of diseased or injured bone is at least temporarily restored to a near normal geometry in order that said near normal geometry may be more permanently restored.

“Discectomy” refers to the partial or complete removal of a diseased or traumatized intervertebral disc.

A system that allows access to either or both the vertebral body or the disc space above and/or below the vertebral body is disclosed. The system may comprise a steerable cutting device for creating a path through bone to allow access for other devices to deliver a therapy, such as, for example, removing tissue such as the nucleus of an intervertebral disc, and/or delivering a material and/or a prosthesis where tissue has been removed. The steerable cutting device may comprise a laser, ultrasound, high speed burr, vibrating tip, drill bit, or other suitable cutting means and may be housed within a compliant polymer catheter having support ribs.

Through a small external incision, the device is introduced into the vertebral body in a posterior-lateral, transpedicular fashion, penetrating the vertebral body using suitable means. Once within the vertebral body, the distal end of the device may be directed via the steering means to cut a curved path in the vertical plane, either superiorly into the disc space above the vertebral body, or inferiorly into the disc space below the vertebral body, in order to create a path through the vertebral body to allow access to subsequent devices to perform procedures on the disc. Alternatively, the device may create a path within the vertebral body in order to allow access to subsequent devices to treat the vertebral body itself. Also in the alternative, the device can be directed to curve in the horizontal plane to create a path along the anterior curvature of the interior of the vertebral body in order to allow access to devices to perform procedures within the vertebral body. Once a desired path is cut using the steerable cutting means, the steerable cutting means can be withdrawn and the desired therapy may be introduced into either the vertebral body or the disc space via the desired path. The desired therapy may include, for example, a biofoam filler for repair of the vertebral body. Alternatively, the therapy may comprise, for example, a biofoam filler such as curable polyflouropolyether within a membrane to serve as a nucleus replacement. The membrane may comprise partitions to maintain materials of varying moduli within desired parts of the device and in order to more closely mimic the mechanical properties of a healthy native nucleus. As a further example, materials of varied moduli may be selected depending upon a patient's needs and/or the position of the particular vertebral body undergoing treatment. For example, a higher modulus material or materials may be selected for an older patient or patient for whom stability is the highest priority; a lower modulus material or materials may be more advantageous for a younger and/or more active patient. Or, a higher modulus material may be selected for treatment of a vertebral body and/or disc in the lumbar region of the spine. Conversely, a relatively lower modulus material may be selected for treatment of a vertebral body and/or disc in the cervical region of the spine.

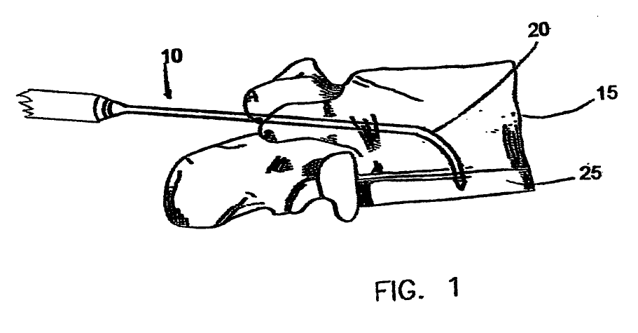

For example, as illustrated in FIG. 1, system 10 can be introduced into vertebral body 15 transpedicularly, and be directed to cut a path (occupied in FIG. 1 by compliant catheter 20) that curves in an inferior manner generally in the vertical plane and extends into the disc space 25 below vertebral body 15, to allow access to a subsequent device in order to remove tissue and/or deliver a therapy. Such an approach may be used where the nucleus pulposus is protruding beyond the disc space (not pictured). The nucleus can then be extracted. Subsequently, using the same path, an artificial nucleus may be delivered and deployed. Any of a number of suitable artificial nucleus devices can be delivered. In a treatment involving a filled membrane, the distal end of the device may comprise a pressure regulator to prevent overexpansion of the membrane. Excess filling material may return to the distal end of the device. More specific examples of devices and methods for treating or replacing intervertebral discs are set forth in provisional U.S. patent application Ser. Nos. 60/535,954, and 10/990,158 and are incorporated herein.

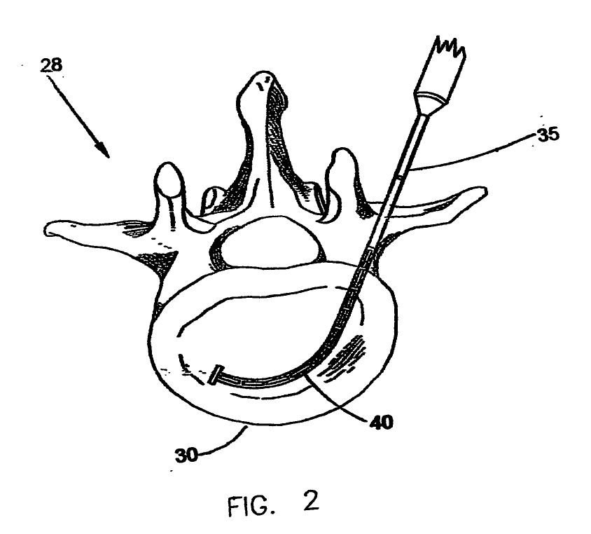

In a second example, as illustrated in FIG. 2, the device may be introduced into vertebral body 28 and used to create a path generally in the horizontal plane, along the anterior curvature 30 of vertebral body 28. Compliant catheter 35 may then be introduced to deliver and deploy support structures 40 within vertebral body 28. Optionally, a filling material or therapeutic device (not pictured) may be introduced either alone or in addition to support structures 40. A filling material may be introduced under controlled pressure. More specific examples of methods and devices for treating a vertebral body are set forth in provisional U.S. patent application Ser. No. 60/504,333 and are incorporated herein.

While particular forms of the invention have been illustrated and described above, the foregoing descriptions are intended as examples, and to one skilled in the art it will be apparent that various modifications can be made without departing from the spirit and scope of the invention.

1. A system for use in the treatment of a disorder of a vertebral body, an intervertebral disc, a spinal unit, or any portion thereof, the system comprising a steerable cutting means for creating a path within or through a vertebral body to allow access for a treatment.

2. The system according to claim 1 further comprising a means for delivering a therapy.

3. The system according to claim 1 wherein said therapy comprises a prosthesis.

4. The system according to claim 2 wherein said therapy comprises a filling material.

5. The system according to claim 1 wherein said treatment is selected from the group consisting of partial or complete discectomy, partial or total disc replacement, vertebroplasty, vertebral decompression, prosthesis delivery, and prosthesis deployment.

6. A method for obtaining access to the interior of a vertebral body or to the intervertebral body disc space, said method comprising accessing the interior of a vertebral body from a transpedicular approach.

7. The method according to claim 6 wherein said method further comprises creating a path within the vertebral body.

8. The method according to claim 7 wherein said path is generally oriented along the horizontal plane.

9. The method according to claim 7 wherein said path is generally oriented in the vertical plane.

10. The method according to claim 9 wherein said method comprises the additional step of entering the intervertebral disc space either above or below the vertebral body.

11. A method for treating a spinal disorder comprising obtaining access to the interior of a vertebral body from a transpedicular approach.

12. The method according to claim 11 wherein the method further comprises creating a path within said vertebral body.

13. The method according to claim 12 wherein said path is generally oriented in the horizontal plane.

14. The method according to claim 12 wherein said path is generally oriented in the vertical plane.

15. The method according to claim 12 wherein the method further comprises the step of delivering a prosthesis within the vertebral body.

16. The method according to claim 15 wherein the method further comprises the step of deploying the prosthesis within the vertebral body.

17. The method according to claim 12 wherein the method further comprises the step of introducing a filling material within the vertebral body.

18. The method according to claim 12 wherein the method further comprises the step of entering the intervertbral disc space.

19. The method according to claim 18 wherein the method further comprises the step of extracting some or all of the native disc.

20. The method according to claim 19 wherein the method further comprises the step of delivering a prosthesis to the intervertebral disc space.

21. The method according to claim 20 wherein the method further comprises the step of deploying the prosthesis.

22. The method according to claim 21 wherein the step of deploying the prosthesis comprises introducing a filling material into the prosthesis.

23. The system according to claim 1, wherein said means for penetrating a vertebral body comprises a high speed burr.

24. The system according to claim 1, wherein said means for penetrating a vertebral body comprises a vibrating tip.

25. The system according to claim 1, wherein said means for penetrating a vertebral body comprises a drill bit.

26. The system according to claim 1, said system further comprising a pressure regulator to control the delivery of a therapy.

27. The system according to claim 26, wherein said therapy comprises a curable polymeric foam.

28. The system according to claim 1, wherein said treatment comprises removal of tissue.

29. The system according to claim 28, wherein said treatment comprises removal of some or all of the native disc.

30. The system according to claim 1, wherein said means for penetrating a vertebral body comprises a flexible drill bit.

31. The system according to claim 1, wherein said means for penetrating a vertebral body comprises ultrasound.

32. The method according to claim 7 wherein said path is generally oriented between the vertical and the horizontal plane.

33. The method according to claim 12 wherein said path is generally oriented between the vertical and the horizontal plane.
